(19) United States
(12) Patent Application Publication
(10) Pub. No.: US 2014/0368796 A1
(43) Pub. Date: Dec. 18, 2014

(54) OPHTHALMOLOGIC APPARATUS, AND METHOD OF CONTROLLING THE SAME

(71) Applicant: CANON KABUSHIKI KAISHA, Tokyo (JP)

(72) Inventor: Hiraku Ogura, Tokyo (JP)

(21) Appl. No.: 14/297,071

(22) Filed: Jun. 5, 2014

(30) Foreign Application Priority Data
Jun. 17, 2013 (JP) 2013-126728

(51) Int. Cl. A61B 3/00

(52) U.S. Cl. CPC A61B 3/0075
USPC 351/245; 351/246

(57) ABSTRACT

There is provided an electric joystick for an ophthalmologic apparatus which exhibits a relatively small difference from operation of a manual joystick when performing alignment of an ophthalmologic apparatus. An ophthalmologic apparatus including an inspection unit which images or measures part of an eye to be inspected, a driving unit which moves the inspection unit, and an operation knob tiltable in an arbitrary direction includes a detection unit which detects a detected amount corresponding to the operation force applied to the operation knob in a case where the tilt angle of the operation knob tilted from a non-tilt position exceeds a predetermined angle, and a control unit which drives the driving unit in accordance with the detected amount detected by the detection unit.

### BACKGROUND OF THE INVENTION

1. Field of the Invention

The present invention relates to an ophthalmologic apparatus and, more particularly, to an ophthalmologic apparatus which inspects, observes, and images an eye to be inspected upon alignment of a detection portion with the eye using a joystick, a method of controlling the apparatus, and a program.

2. Description of the Related Art

Many of the ophthalmologic apparatuses include a base portion including a face rest which fixes the face of an object to be examined, a detection portion which performs observation, imaging, measurement, and the like of an eye to be inspected, and a stage portion which moves the detection portion forward, backward, leftward, rightward, upward, and downward relative to the base portion. Such an apparatus includes a joystick mechanism to be operated to drive the stage portion. The joystick used by the ophthalmologic apparatus needs to have both functions for fine motion operation required for alignment with an eye to be inspected and coarse motion operation required for changing between the left and right eyes.

Many conventional ophthalmologic apparatuses have been using a manual stage which mechanically links the joystick to the stage portion, and mechanically driving the detection portion with the joystick (to be referred to as a manual joystick hereinafter). There is known, as a manual joystick, a joystick of a scheme designed to move a detection portion by making an examiner tilt an operation knob about the contact point, as an operation fulcrum, between a hemispherical support member placed under the operation knob and a friction plate placed on the apparatus base side. The operation knob can tilt in all directions centered on a neutral point. It is possible to freely move the detection portion based on the tilt angle and direction of the operation knob. When it is necessary to finely move an inspection portion to align an eye to be inspected with the detection portion, the examiner moves the inspection portion and al it with the eye by adjusting the angle (to be referred to as the tilt angle hereinafter) at which the operation knob tilts. When changing between the left and right eyes, the examiner can slide the pointing member of the operation knob on a friction plate by pushing the joystick in a desired moving direction and easily implement coarse motion operation.

Recently, there are an increasing number of ophthalmologic apparatuses which include an electric stage driven by a motor or the like owing to advantages such as automatic alignment. Unlike a conventional joystick mechanism, the electric stage cannot be moved by using a mechanical link. For this reason, such an apparatus includes an electric joystick capable of performing control using electrical signals as a drive instruction input device for an electric stage portion. As an ophthalmologic apparatus including such an electric stage and electric joystick, there is known an ophthalmologic apparatus which improves the operability of an electric joystick by detecting an operation speed when the operation knob is tilted. Such an ophthalmologic apparatus exemplified in Japanese Patent Application Laid-Open No. 2009-56247 includes a control mechanism which can detect an operation speed when the operation knob is tilted, and changes the moving amount of the detection portion in accordance with the magnitude of the operation speed. When the operation knob is tilted to the tilt limit, the ophthalmologic apparatus detects that the tilt angle has reached the limit position, and coarsely moves the detection portion.

The ophthalmologic apparatus disclosed in Japanese Patent Application Laid-Open No. 2009-56247 is configured to change the coarse motion speed by a speed adjusting switch, resulting in a larger speed change difference than when driving the detection portion by operating the manual joystick. That is, the manual joystick can continuously adjust the speed of the detection portion in accordance with the adjustment of force to push the operation knob, whereas the electric joystick using the speed adjusting switch is designed to perform intermittent speed adjustment. That is, the manual joystick and the electric joystick give the examiner different maneuvering feelings because of the differences in operation method and detection portion behavior based on operations. For this reason, an ophthalmologic apparatus using an electric joystick is required to improve the operability of the electric joystick, including bringing the operation method and the behavior of the detection portion based on operations near to those of a manual joystick, in other words, bringing the operation characteristics of the electric joystick near to those of the manual joystick.

### SUMMARY OF THE INVENTION

The present invention has been made in consideration of the above situation, and provides an electric joystick which can obtain a maneuvering feeling similar to that of a manual joystick according to a related art.

In order to solve the above problem, an ophthalmologic apparatus according to the present invention comprises an inspection unit configured to inspect an eye to be inspected, a driving unit configured to move the inspection unit, a detection unit configured to detect an amount corresponding to an operation force applied to an operation unit in a case where a tilt angle of the operation unit tilted from a non-tilt position exceeds a predetermined angle, and a control unit configured to control the driving unit in accordance with the detected amount.

The ophthalmologic apparatus according to the present invention can detect the operation force applied by the examiner to tilt the operation knob at the time of coarse motion operation. Controlling the speed of the detection portion by using the detected operation force allows the examiner to change the moving speed of the detection portion by adjusting a force to push the operation knob. This makes it possible to obtain, even with the electric joystick, a maneuvering feeling similar to that of a manual joystick according to a related art.

Further features of the present invention will become apparent from the following description of exemplary embodiments with reference to the attached drawings.

### BRIEF DESCRIPTION OF THE DRAWINGS

FIG. 1 is a schematic view showing the overall arrangement of an ophthalmologic apparatus according to an embodiment of the present invention.

FIG. 2 is a perspective view showing the outer appearance of a joystick provided for the ophthalmologic apparatus shown in FIG. 1.

FIG. 3 is a sectional view of the joystick shown in FIG. 2.

FIG. 4 is a view for explaining an operation force generated by the joystick.

FIG. 5 is a system block diagram of the ophthalmologic apparatus according to the embodiment of the present invention.

FIGS. 6A, 6B and 6C are views showing the principle of detecting the operation force generated by the joystick.

FIG. 7 is a flowchart showing control performed by a detection portion.

FIGS. 8A and 8B are views showing the content of control corresponding to the tilt angle of the joystick.

FIGS. 9A and 9B are views showing the contents of control corresponding to the tilt operation force of the joystick.

### DESCRIPTION OF THE EMBODIMENTS

Preferred embodiments of the present invention will now be described in detail in accordance with the accompanying drawings.

### First Embodiment

Arrangement

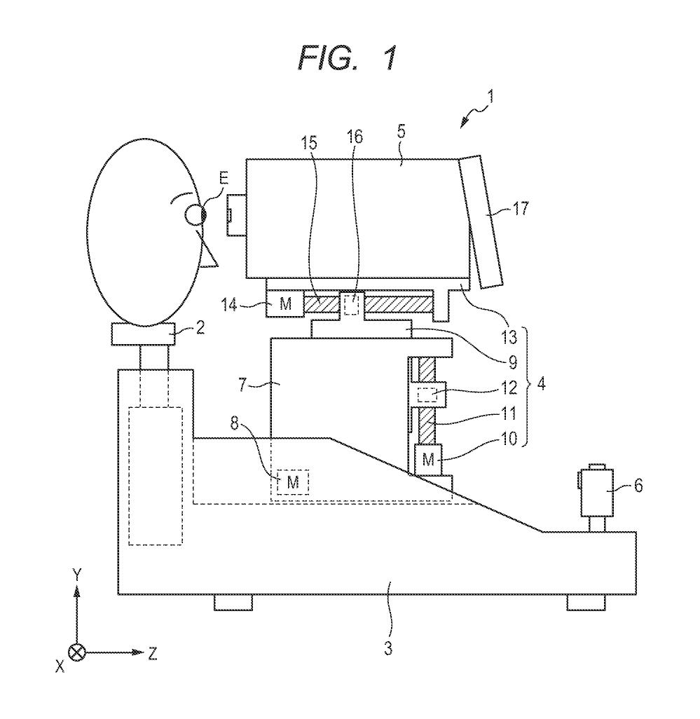

FIG. 1 is a schematic view showing the overall arrangement of an ophthalmologic apparatus 1 according to the first embodiment of the present invention.

Overall Arrangement

The ophthalmologic apparatus 1 includes, as main components, a base 3 having a face rest 2 which supports the face of an object to be examined, a driving portion 4 provided on the base 3, a detection portion 5 mounted on the driving portion 4, and an electric joystick 6 as an operation member.

Three-Axis Stage

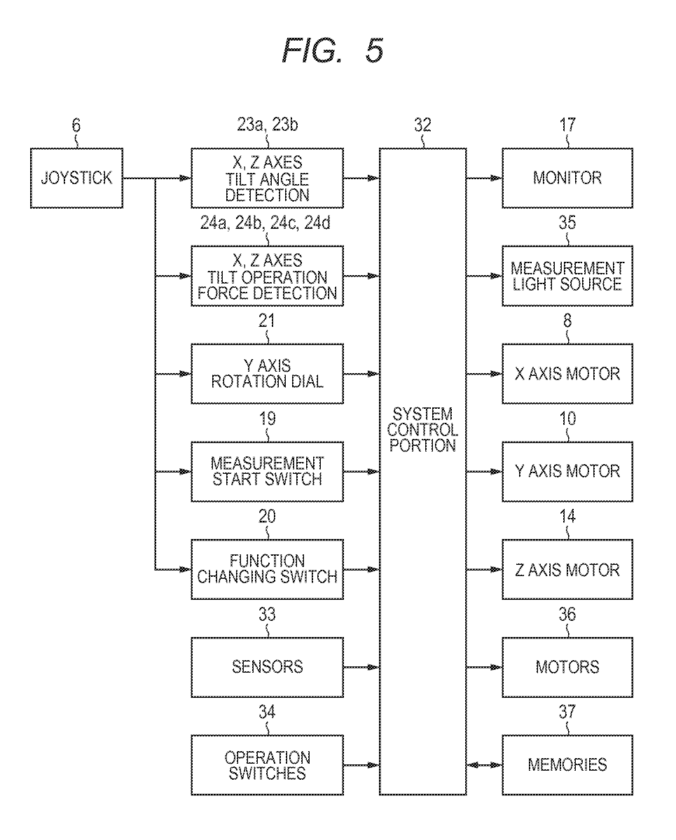

The driving portion 4 is formed as a three-axis stage which drives the detection portion 5 in the three axes directions, namely the X, Y, Z axes directions, relative to the base 3, and functions as a driving unit which moves an inspection unit. Referring to FIG. 1, a frame 7 is movable in the horizontal direction (the direction parallel to the drawing surface to be referred to as the X axis direction hereinafter) relative to the base 3. A driving mechanism in the X axis direction includes an X axis driving motor 8 fixed on the base 3, a feed screw (not shown) coupled to the motor output shaft of the motor, and a nut (not shown) fixed to the frame 7 which can move on the feed screw in the X axis direction. At the time of the operation of the frame 7, the X axis driving motor 8 operates in accordance with a rotation signal from a system control portion 32 (see FIG. 5) to rotate the feed screw. This rotating operation through the nut then moves the frame 7 in the X axis direction.

Likewise, a frame 9 is movable in the vertical direction (the direction perpendicular to the drawing surface, to be referred to as the Y axis direction hereinafter) relative to the frame 7. A driving mechanism in the Y axis direction includes a Y axis driving motor 10 fixed on the frame 7, a feed screw 11 coupled to the motor output shaft of the motor, and a nut 12 fixed to the frame 9 which can move on the feed screw in the Y axis direction. At the time of the operation of the frame 9, the Y axis driving motor 10 operates in accordance with a rotation signal from the system control portion 32 to rotate the feed screw 11. This rotating operation through the nut 12 then moves the frame 9 in the Y axis direction.

In addition, a frame 13 is movable in the back and forth direction (the left and right direction on the drawing surface to be referred to as the Z axis direction hereinafter) relative to the frame 9. A driving mechanism in the Z axis direction includes a Z axis driving motor 14 fixed on the frame 13, a feed screw 15 coupled to the motor output shaft of the motor, and a nut 16 fixed to the frame 9 which can move on the feed screw in the Z axis direction. At the time of the operation of the frame 13, the Z axis driving motor 14 operates in accordance with a rotation signal from the system control portion 32 to rotate the feed screw 15. This rotating operation through the nut 16 then moves the frame 13 in the Z axis direction.

The detection portion 5 can be independently driven along the X, Y, and Z axes owing to the driving principles about the respective axes described above. In addition, the system control portion 32 determines the operation amounts of the motors of the respective axes (the X axis driving motor 8, the Y axis driving motor 10, and the Z axis driving motor 14) based on input signals (to be described later) from the joystick 6.

Note that in this embodiment, the detection portion 5 is configured to be driven in the Y axis direction based on the electrical signal obtained from the joystick 6. However, according to the feature of the present invention, the detection portion 5 is electrically driven leftward, rightward, forward, and backward within a two-dimensional plane. A scheme of driving the detection portion 5 in the vertical direction is not limited to an electric driving scheme. For this reason, the present invention can be effectively applied to an ophthalmologic apparatus having an arrangement based on a mechanical driving scheme for driving in the vertical direction typified by a belt transmission mechanism.

Detection Portion

The detection portion 5 for performing measurement is fixed on the frame 13. The detection portion 5 functions as an inspection unit which images or measures part of an eye to be inspected. Note that the contents of inspection by the detection portion 5 will be collectively termed optometry or optometric operation for obtaining various types of ocular characteristics concerning an eye to be inspected, including these imaging and measuring operations. For example, in a non-mydriatic fundus camera, a detection portion incorporates a fundus illumination optical system including an observation light source for irradiating an eye to be inspected with light at the time of observation and an imaging light source used at the time of imaging, an observation imaging optical system for forming reflected light from the eye into an image on an imaging element (not shown), and the like. In this case, reflected light from an eye to be inspected which is illuminated by the fundus illumination optical system is formed into an image on the imaging element through the observation imaging optical system. A monitor 17 as a display member then displays the fundus image. Alternatively, an image memory (not shown) stores the image as a still image. Note that this embodiment exemplifies the non-mydriatic fundus camera. However, the ophthalmologic apparatus to which the present invention is applied is not limited to this. The present invention can be applied to various types of ophthalmologic apparatuses used for ophthalmologic examination, including an OCT apparatus and an AO-SLO apparatus.

Joystick

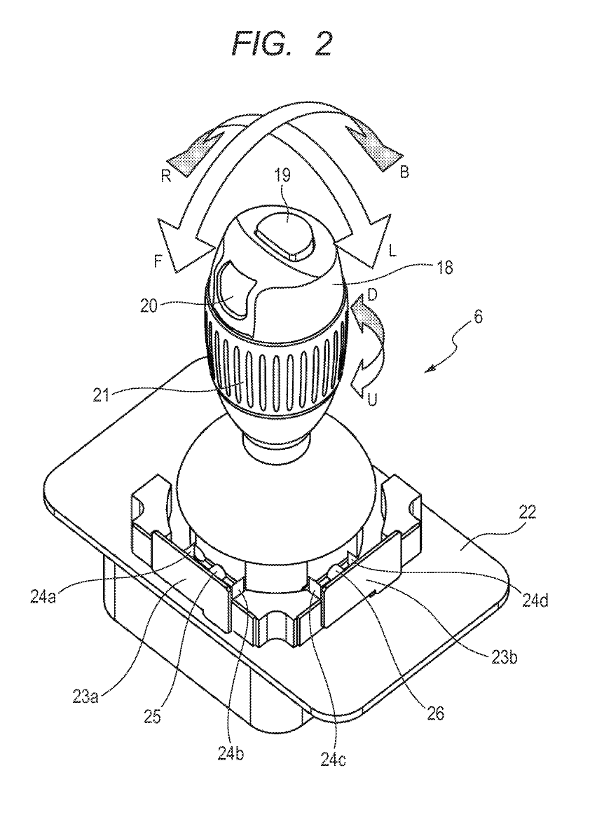

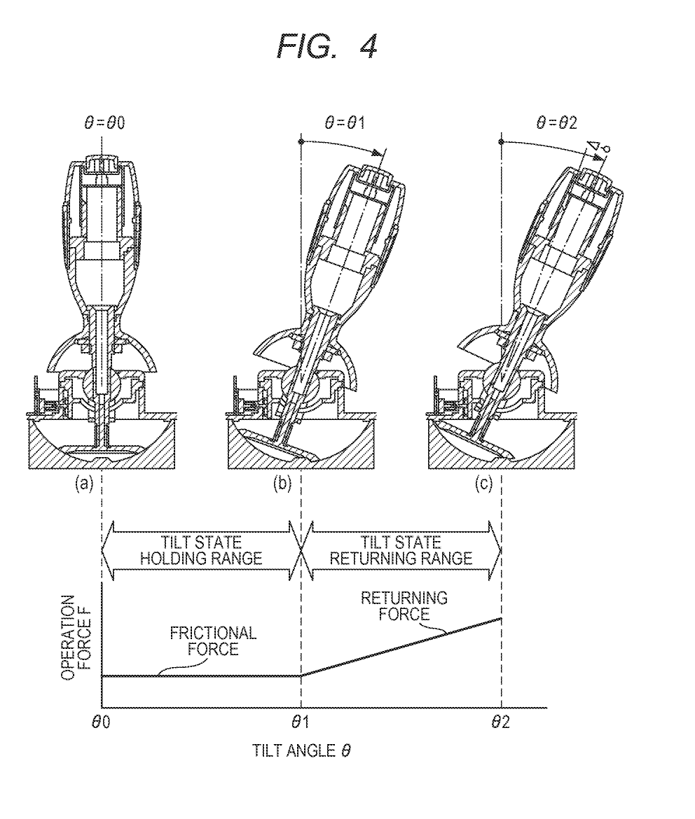

FIG. 2 is a perspective view of the electric joystick 6 including an operation knob which can tilt in an arbitrary direction, which is used in the embodiment shown in FIG. 1. Note that a tilt angle θ to be described below corresponds to a tilt angle relative to a neutral position at which an operation knob 18 has no tilt as shown in FIG. 4. In addition, an operation force is a force applied by the examiner to bias the operation knob 18 to tilt it. The joystick 6 includes the operation knob 18, a measurement start switch 19, a function changing switch 20, a rotation dial 21, a bearing base 22, tilt angle detection portions 23a and 23b, operation force detection portions 24a to 24d, and slide pins 25 and 26. The tilt angle detection portions 23a and 23b function as detection portions (units) which detect the tilt angle of the operation knob. The operation force detection portions 24a to 24d function as detection portions (units) which detect the operation force to tilt the operation knob. The slide pins 25 and 26 move in the horizontal direction in synchronism with the tilting of the operation knob. In this case, the slide pins 25 and 26 are respectively joined to the input shafts of the tilt angle detection portions 23a and 23b. In addition, the operation force detection portions 24a and 24b are placed to be pressed by the slide pin 25 when the slide pin 25 translates and reaches the end of its moving range. Likewise, the operation force detection portions 24c and 24d are placed to come into contact with and be pressed by the slide pin 26 at the end of the moving range. Note that the detection portions will be described in detail later.

The electric joystick 6 is configured to issue a drive instruction based on the driving direction, driving amount, and driving speed of the driving portion 4 and move the detection portion 5 in three-dimensional directions. When the examiner tilts the operation knob 18 as a member for performing various types of operations in the direction indicated by a double-headed arrow LR, the detection portion 5 moves in the eye width direction (X axis direction) of an eye to be inspected. When the examiner tilts the operation knob 18 in the direction indicated by a two-way direction arrow FB, the detection portion 5 moves in a direction (Z axis direction) to approach or separate from the eye. When the examiner rotates the rotation dial 21 in the direction indicated by a double-headed arrow UD, the detection portion 5 moves in the vertical direction (Y axis direction). The measurement start switch 19 is placed above the operation knob 18. When the examiner presses the measurement start switch 19, the apparatus starts inspection, observation, imaging, and automatic alignment. The function changing switch 20 is used to change between a plurality of operation modes implemented in the ophthalmologic apparatus. For example, in a fundus camera, this switch is used to change from an anterior ocular segment observation state of an eye to be inspected to a fundus observation state. This allows the examiner to perform operation from alignment to measurement only by operating the joystick 6. Note that the operation knob 18 is an aspect of an operation unit of the present invention and can be replaced with units providing the same functions as the operation knob.

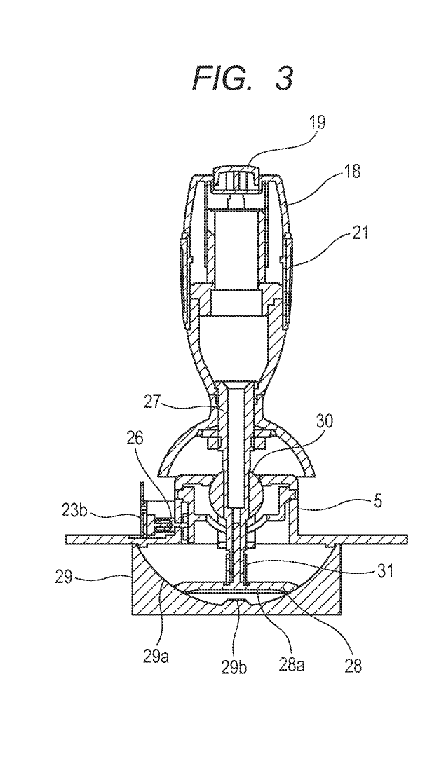

FIG. 3 is a sectional view of the electric joystick 6 shown in FIG. 2 when taken along the direction indicated by the double-headed arrow LR. Referring to FIG. 3, a center ball 30 is fixed at a predetermined position of an operation knob shaft 27, and a moving portion 28 is placed below the operation knob shaft 27. A hollow portion is formed in the lower end side of the operation knob shaft 27. The central shaft of the moving portion 28 is fitted in the operation knob shaft 27. The moving portion 28 is slideable in the direction of the operation knob shaft 27 relative to the operation knob shaft 27. A disk-like shape having a concave portion 28a with a concave shape in a central portion is formed below the central shaft of the moving portion 28. An operation force generating portion joined to the bearing base 22 is provided below the moving portion 28. A nearly spherical surface 29a centered on the curvature center of the center ball 30 is formed on the operation force generating portion 29. A returning member having a convex portion 29b with a convex shape is formed on the central portion of the nearly spherical surface 29a. A compression spring 31 as an elastic member is provided between the operation knob shaft 27 and the moving portion 28. The compression spring 31 generates a biasing force by being compressed, and biases the center ball 30 against the bearing base 22 with the biasing force. At the same time, the compression spring 31 biases the moving portion 28 against the operation force generating portion 29, especially, the spherical surface 29a and the convex portion 29b.

The operation of the joystick 6 when the operation knob 18 is tilted will be described with reference to FIG. 4. Like FIG. 3, FIG. 4 is a sectional view of the joystick 6 when taken along the direction indicated by a double-headed arrow RL.

A state (a) in FIG. 4 indicates the neutral state of the operation knob 18. A state (b) in FIG. 4 indicates a state in which the operation knob 18 is tilted from the neutral position indicated in the state (a) to a predetermined angle θ1. A state (c) in FIG. 4 indicates a state in which the operation knob 18 is tilted from the neutral position to a predetermined angle θ2. In this case, the predetermined angle θ1 is the maximum tilt angle in a tilt state holding range, and the predetermined angle θ2 is the maximum tilt angle in a tilt state returning range.

A case in which the operation knob 18 is tilted in the tilt state holding range (between the tilt angle θ0 and the tilt angle θ1) will be described first. In the tilt state holding range, the compression spring 31 biases the moving portion 28 against the nearly spherical surface 29a of the operation force generating portion 29. At this time, a frictional force is generated between the moving portion 28 and the operation force generating portion 29. With this frictional force, the tilt angle of the operation knob 18 can be held. In addition, since the nearly spherical surface 29a of the operation force generating portion 29 is formed with a curvature centered on the tilt center, the compression spring 31 does not expand and contract within the tilt state holding range in spite of movement in an arbitrary direction. For this reason, it is possible to generate constant frictional force regardless of the operating direction of the operation knob 18. This makes it possible to hold the operation force constant in arbitrary directions.

A case in which the operation knob 18 is tilted to the tilt state returning range will be described next. When the examiner tilts the operation knob 18 to the tilt state returning range, the inclined surface formed on the outer circumference of the concave portion 28a of the moving portion 28 comes into contact with the returning portion (convex portion) 29b of the operation force generating portion 29 (see the state (b) in FIG. 4). When the examiner further tilts the operation knob 18, the moving portion 28 moves in the axial direction (obliquely upper right in the state (b) in FIG. 4) owing to a component of the force received from the inclined surface in the axis direction of the operation knob shaft 27 (see state (c)). At this time, the compression spring 31 is compressed. In this case, since an operation force corresponding to the degree of compression of the compression spring 31 is required, the examiner can recognize that he/she has operated the operation knob 18 up to the tilt state returning range. At this time, when the examiner releases the operation knob 18, the operation knob 18 returns to the predetermined angle θ1 indicated by state (b) in FIG. 4 owing to the returning force. Since the compression spring 31 expands and contracts in proportion to the tilt angle of the operation knob 18, the returning force generated by the compression spring 31 is also proportional to the tilt angle of the operation knob, as shown in FIG. 4.

The above components constitute a returning force generation unit in this embodiment which biases the operation knob 18 with the returning force to return the operation knob 18 to a predetermined angle in accordance with the difference between a predetermined angle and a tilt angle. The returning force generating unit generates a returning force to return the operation knob 18 to a predetermined angle or into a predetermined angle range by making the moving portion 28 connected to the operation knob 18 abut against the operation force generating portion 29 as an abutment surface against which the moving portion 28 abuts, and also making the moving portion 28 ride on the convex portion 29b. In other words, the moving portion 28 is connected to an end portion of the operation knob 18 so as to be movable in the axial direction of the operation knob shaft 27, and the operation force generating portion 29 as the above abutment surface has the spherical surface 29a which slideably abuts against the moving portion 28, centered on the center of the center ball 30 as the operation center of the operation knob 18. The returning force generating unit then generates a returning force by making the convex portion 29b abut against the moving portion 28 by using the elastic member 31 which biases the operation knob 18 and the moving portion 28 in a direction to separate them from each other and presses the moving portion 28 against the spherical surface 29a.

As described above, the operation knob 18 generates the frictional force and reaction force of returning force like those shown in FIG. 4 in accordance with the tilt angle of the operation knob 18. The examiner can change the tilt angle of the operation knob 18 by applying an operation force to this reaction force.

System Control Portion

FIG. 5 is a system block diagram of the ophthalmologic apparatus 1.

The ophthalmologic apparatus 1 functions as an ophthalmologic apparatus which inspects an eye to be inspected by being controlled by the system control portion 32. The joystick 6, an input portion, an output portion, and memories 37 are connected to the system control portion 32. The input portion includes various types of sensors 33 and various types of operation switches 34. The output portion includes the monitor 17, a measurement light source 35, the X axis driving motor 8, the Y axis driving motor 10, the Z axis driving motor 14, and various types of motors 36. The system control portion 32 performs detection of various types of input signals, analysis of the input signals, and control of various types of outputs.

The various types of sensors 33 include a limit sensor which detects the limit of the movement of the driving portion. The various types of operation switches include a switch for allowing the examiner to make various types of settings for the apparatus. The measurement light source 35 is a light source which illuminates an eye E to be inspected for observation and imaging. The various types of motors 36 include a motor for adjusting the height of the face rest 2 and a motor for driving the optical system formed inside the detection portion 5. The memories 37 include a memory capable of writing and reading out various types of data.

Operation

A method of controlling the detection portion 5 when the examiner has operated the joystick 6 will be described next in detail.

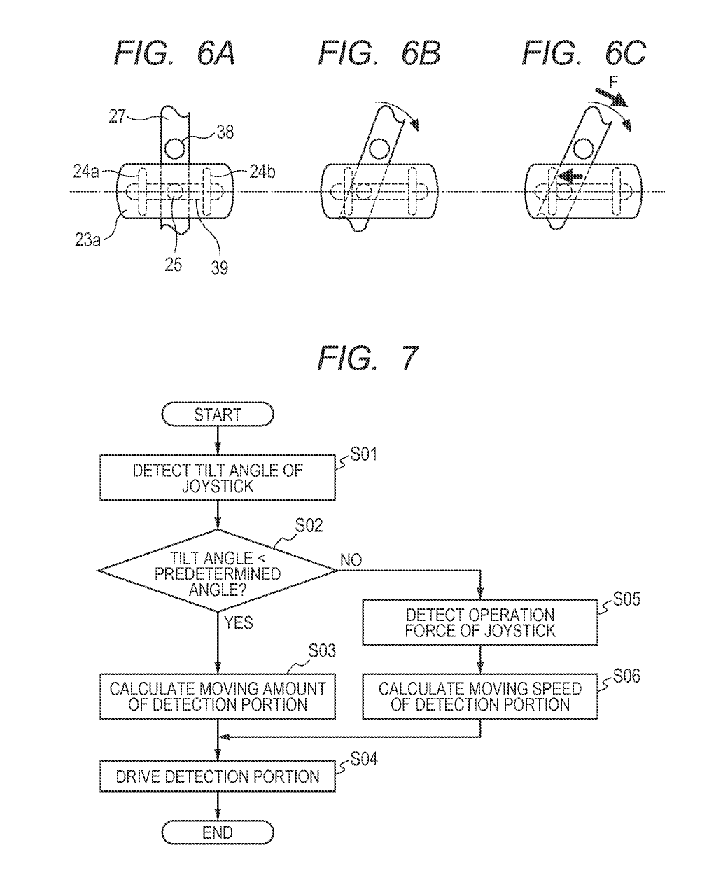

A state (a) in FIGS. 6A, 6B and 6C indicate the positional relationship between a rotational center shaft 38, a groove portion 39, the slide pin 25, the tilt angle detection portion 23a, and the operation force detection portions 24a and 24b as components inside the operation knob portion at the neutral position. When the examiner tilts the operation knob 18 in the X axis direction, the operation knob shaft 27 provided in the operation knob portion also tilts about the rotational center shaft 38 as indicated by a state (b) in FIGS. 6A, 6B and 6C. Accompanying this operation, the the slide pin 25 fitted in the groove portion 39 provided in the operation knob shaft 27 translates in the X axis direction. Since the slide pin is coupled to the input shaft of the tilt angle detection portion 23a, an input to the tilt angle detection portion 23a changes as the operation knob tilts, thereby enabling the detection of an operation amount. Note that this embodiment uses the tilt angle detection portion 23a as a direct acting type potentiometer and detects the tilt angle of the operation knob 18 by outputting the resistance value of the potentiometer to the system control portion 32 via an A/D converter (not shown). Note that the tilt angle detection portion 23a may perform detection by using an optical sensor or magnetic sensor such as a rotary encoder.

When the examiner tilts the operation knob 18 in the Z axis direction, the apparatus detects a tilt angle as in the above case in the X axis direction by using the slide pin 26 and the tilt angle detection portion 23b. For this reason, a detailed description of this operation will be omitted. It is possible to obtain the tilt state of the operation knob 18 in an arbitrary direction as resistance values of an X axis component and Z axis component in the respective axis directions by using the tilt angle detection portion 23a and the tilt angle detection portion 23b having the above arrangements. Reading these resistance values via the system control portion 32 can uniquely detect the tilt direction and angle of the operation knob 18.

A state (c) in FIGS. 6A, 6B and 6C indicate a case in which the tilt angle of the operation knob 18 is further increased. When the examiner tilts the operation knob 18 from the neutral position and the slide pin 25 moves by a predetermined distance, the slide pin 25 comes into contact with the operation force detection portion 24a placed vertical to the moving direction of the slide pin 25. At this time, when the examiner operates the operation knob 18 in a direction to further increase the tilt angle, the operation force detection portion 24a is greatly pressed in accordance with an operation force F. When the examiner decreases the operation force F, the force to press the operation force detection portion 24a also decreases. Therefore, placing a pressure sensor and the like as the operation force detection portion 24a to form an operation force detection unit can detect a pressure corresponding to the force applied by the examiner to tilt the operation knob 18. That is, the operation force detection portion 24a obtains, as a detected amount, the operation force F applied to the operation knob 18 against the returning force to return the operation knob 18 to a predetermined angle. In this case, the operation force detection portion 24a functioning as a detection portion is formed from a pressure sensor which receives a pressing force from the operation knob 18. However, as will be described later, this detection portion may obtain the operation force from other parameters, for example, may calculate the operation force from the tilt angle of the operation knob. In this case, the detected amount obtained by the detection portion corresponds to operation force.

The detection units for a tilt angle and operation force are arranged in the above manner to detect the tilt angle and operation force of the operation knob 18, thereby controlling the detection portion 5 in accordance with the operation performed on the operation knob 18. Although this embodiment uses the same detection method with respect to the X and Z axes as described above, it is possible to control the detection portion 5 concerning the X and Z axes by the same method. For the sake of easy understanding, the following control method will be described concerning only the X axis.

FIG. 7 is a flowchart showing control performed by the system control portion 32 until the detection portion 5 is driven upon tilting of the operation knob 18. When the examiner tilts the operation knob 18 in the X axis direction to align the eye E with the detection portion 5, an output value from the tilt angle detection portion 23a is sent to the system control portion 32 to detect the tilt angle of the operation knob 18 (step S01). Subsequently, in step S02, the system control portion 32 compares the detected tilt angle with a predetermined angle to determine which is larger. In this case, the predetermined angle corresponds to the maximum tilt angle in the tilt state holding range.

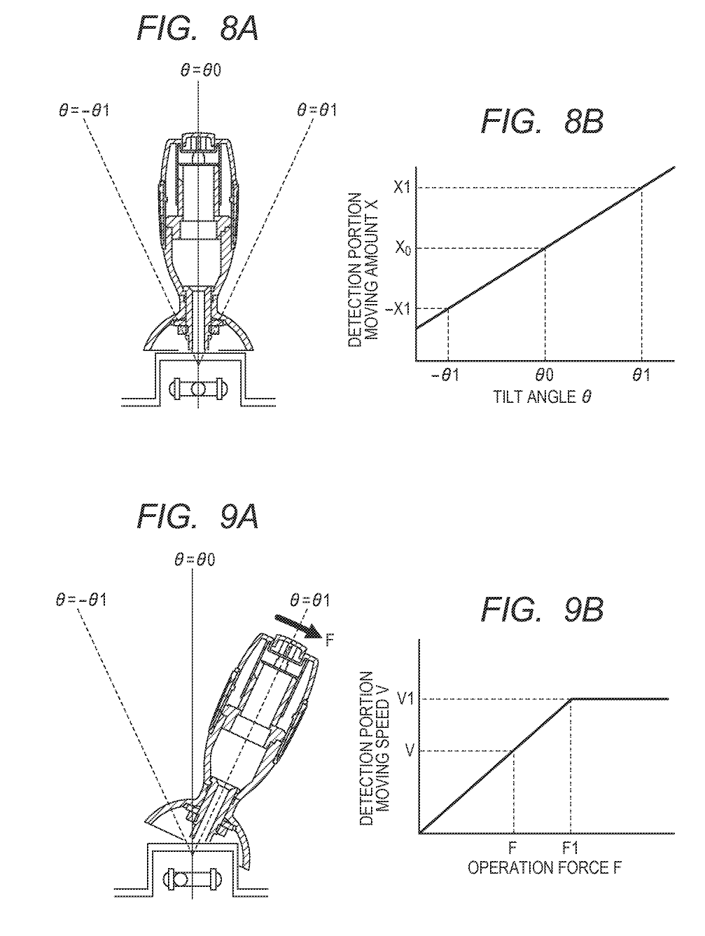

If the tilt angle is equal to or less than the predetermined angle, the process advances to step S03 to calculate the moving amount of the detection portion 5 in accordance with the tilt angle of the operation knob 18 detected by the detection portion 23a. The tilt angle detected by the detection portion 23a is converted into the moving amount of the detection portion 5 in the X axis direction by the system control portion 32 according to the relationship shown in FIGS. 8A and 8B, thereby calculating the moving amount of the detection portion 5. FIGS. 8A and 8B shows an example of the correspondence relationship between the tilt angle of the operation knob 18 and the moving amount of the detection portion 5 in the X axis direction. The system control portion 32 rotates the X axis driving motor 8 to move the detection portion 5 by the calculated moving amount (step S04). The system control portion 32 can control the position of the detection portion 5 in accordance with the tilt angle of the operation knob 18 by repeating the above steps, and can finely move the detection portion 5 to accurately align it with an eye to be inspected.

Assume that the examiner has further tilted the operation knob 18 in the X axis direction, and the the system control portion 32 has determined in step S02 that the tilt angle has exceeded the predetermined angle. In this case, the process advances to step S05. In step S05, the detection portion 24a or 24b detects the operation force F with which the examiner tilts the operation knob 18. A signal representing the operation force detected by the detection portions 24a and 24b is sent to the system control portion 32. In step S06, the system control portion 32 determines the moving speed of the detection portion 5 in proportion to the operation force, as shown in FIGS. 9A and 9B. FIGS. 9A and 9B show an example of the correspondence relationship between the operation force F detected by the operation force detection portions 24a and 24b and a corresponding moving speed V of the detection portion 5. This embodiment exemplifies a case in which the operation force F and the moving speed V are proportional to each other. However, it is possible to control a moving speed in accordance with a detected amount as needed, for example, by calculating the moving speed of the detection portion by changing a proportionality coefficient in accordance with the magnitude of the operation force F. The system control portion 32 controls the X axis driving motor 8 to move the detection portion 5 at the speed determined by the system control portion 32 (step S04). Note, however, that the driving motor which moves the detection portion 5 has an upper rotational speed limit, and excessively fast movement of the detection portion is not preferable in terms of safety. For these reasons, an upper limit value is set for the moving speed. Performing the above control can adjust the moving speed of the detection portion 5 in accordance with the magnitude of a force to tilt, even when the examiner greatly tilts the operation knob 18 to coarsely move the detection portion 5.

With the above control, when wanting to accurately align the detection portion 5 with the eye E, the examiner adjusts the tilt angle of the operation knob 18, whereas when wanting to coarsely move the detection portion 5 to, for example, change between the left and right eyes, the examiner largely tilts the operation knob 18, and can adjust the moving speed of the detection portion 5 by further adjusting the operation force. This can implement an electric joystick with better operability than that in the related art, since the examiner can control the fine and coarse movements of the detection portion just by tilting the operation knob 18. That is, according to this embodiment, when the examiner tilts the operation knob 18, it is possible to detect not only a tilt angle but also the operation force with which the examiner tilts the operation knob 18. Performing speed control on the detection portion 5 by using the detected operation force makes it possible to also adjust the moving speed of the detection portion 5 based on only the tilting operation of the operation knob 18 when coarsely moving the detection portion 5, thereby improving operability.

This embodiment can therefore provide further improved operability as compared with the prior art exemplified which determines a moving speed at the time of coarse motion referring to an operation speed until the operation knob reaches the tilt limit, cannot change the determined speed during coarse motion, and provides the speed adjusting switch to adjust a moving speed during coarse motion.

### Second Embodiment

The first embodiment includes the pressure sensors at the two ends of the slide pin in the moving direction to detect the operation force to tilt the operation knob 18. The second embodiment will describe a method of detecting the operation force without placing any pressure sensor in the arrangement of the electric joystick 6 described in the first embodiment.

The electric joystick includes an operation force generating portion 29 below an operation knob 18 as described in FIG. 3. The operation force generating portion 29 generates a frictional force for holding the tilt angle of the operation knob 18 and a returning force for returning the operation knob 18 in a neutral direction when the tilt angle becomes equal to or more than a predetermined angle. When, therefore, the examiner has tilted the operation knob 18 to a given tilt angle, he/she has applied an operation force corresponding to these frictional and returning forces to the operation knob 18. As described with reference to FIG. 4, the returning force is generated by expansion/contraction of a compression spring 31 caused by the tilting of the operation knob 18, and changes in proportion to the tilt angle of the operation knob 18. It is therefore possible to calculate the returning force generated by the operation force generating portion 29 from the tilt angle of the operation knob 18. This returning force can be used as operation force applied to the operation knob 18 to control a detection portion 5. In this case, a module area functioning as a calculation unit in a system control portion 32 executes the operation of calculating an operation force F as a detected amount in accordance with an increase in tilt angle from a predetermined angle.

Using the above detection method eliminates the necessity to use an operation force detection sensor such as a pressure sensor, and hence can perform coarse control on the detection portion 5 by using the operation knob 18 with a simpler arrangement as compared with the first embodiment.

### Other Embodiments

The present invention is also implemented by executing the following processing. That is, this is the processing of supplying software (programs) for implementing the functions of the above embodiments to a system or apparatus via a network or various types of storage media and making the computer (or the CPU, MPU, or the like) of the system or apparatus read out and execute the software.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-126728, filed Jun. 17, 2013, which is hereby incorporated by reference herein in its entirety.

What is claimed is:

1. An ophthalmologic apparatus comprising:
an inspection unit configured to inspect an eye to be inspected;
a driving unit configured to move the inspection unit;
a detection unit configured to detect an amount corresponding to an operation force applied to an operation unit in a case where a tilt angle of the operation unit tilted from a non-tilt position exceeds a predetermined angle; and
a control unit configured to control the driving unit in accordance with the detected amount.

2. An apparatus according to claim 1, wherein the detected amount is the operation force applied to the operation unit in accordance with a difference between the predetermined angle and the tilt angle.

3. An apparatus according to claim 1, wherein the control unit controls a moving speed of the driving unit in accordance with the detected amount.

4. An apparatus according to claim 1, further comprising a second detection unit configured to detect a second amount different from the detected amount in a case where the tilt angle is not more than the predetermined angle,
wherein the control unit controls the driving unit in accordance with the detected second amount in a case where the tilt angle is not more than the predetermined angle.

5. An apparatus according to claim 4, wherein the second detection unit comprises a unit configured to detect the tilt angle, and the detected second amount is the tilt angle.

6. An apparatus according to claim 4, wherein the control unit controls a moving amount of the driving unit in accordance with the detected second amount in a case where the tilt angle is not more than the predetermined angle.

7. An apparatus according to claim 1, wherein the detection unit comprises a pressure sensor configured to receive a pressing force from the operation unit.

8. An apparatus according to claim 1, wherein the detection unit comprises a calculation unit configured to calculate the detected amount in accordance with an increase in tilt angle from the predetermined angle.

9. An apparatus according to claim 1, further comprising a returning force generating unit configured to bias the operation unit with a returning force to return the operation unit to the predetermined angle in accordance with a difference between the predetermined angle and the tilt angle.

10. An apparatus according to claim 9, wherein the returning force generating unit comprises a moving portion connected to the operation unit and an abutment surface against which the moving portion abuts when the operation unit is operated, and
the returning force is generated when the moving portion rides on a convex portion disposed on the abutment surface.

11. An apparatus according to claim 10, wherein the moving portion is connected to an end portion of the operation unit so as to be movable in an axial direction of the operation unit,
the abutment surface includes a spherical surface which abuts against the moving portion so as to be slideable about an operation center of the operation unit, and
the returning force generating unit generates the returning force by causing the convex portion to abut against the moving portion by using an elastic member configured to press the moving portion against the spherical surface upon biasing in a direction to separate the operation unit from the moving portion.

12. A method of controlling an ophthalmologic apparatus, comprising the steps of:
detecting an amount corresponding to operation force applied to an operation unit, in a case where a tilt angle of the operation unit tilted from a non-tilt position exceeds a predetermined angle; and
controlling a driving unit configured to move an inspection unit configured to inspect an eye to be inspected in accordance with the detected amount.

13. A method according to claim 12, wherein the detected amount is the operation force applied to the operation unit in accordance with a difference between the predetermined angle and the tilt angle.

14. A method according to claim 12, wherein a moving speed of the driving unit is controlled in accordance with the detected amount.

15. A method according to claim 12, further comprising the steps of:
calculating a moving amount of the inspection unit in accordance with the tilt angle if the tilt angle is not more than the predetermined angle; and
causing the driving unit to move the inspection unit by the moving amount.

16. A non-transitory tangible medium having recorded thereon a program for causing a computer to perform steps of the control method according to claim 12.
